(19) United States
(12) Patent Application Publication
(10) Pub. No.: US 2009/0010701 A1
(43) Pub. Date: Jan. 8, 2009

(54) Device For Applying a Gel-like Substance

(76) Inventors: Norbert Minarik, Ruprecht (AT); Georg Achleitner, Graz (AT)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 12/087,855

(22) PCT Filed: Jan. 25, 2007
(86) PCT No.: PCT/AT2007/000031

§ 371 Date: Jul. 16, 2008

(30) Foreign Application Priority Data
Jan. 26, 2006 (AT) GM 52/2006

(51) Int. Cl. A46B 5/02; A61B 8/00

(52) U.S. Cl. 401/6

(57) ABSTRACT

A device for applying a gel-like substance, for instance a contact or ultrasonic gel, includes a gel container having an immersion tube departing from a docking area and extending into the gel-like substance; and an applicator for the gel-like substance mounted in the docking area, the applicator having a reservoir connected to an outlet nozzle as well as a pumping means for the gel-like substance. A tightly sealed connection can be established between the outlet nozzle of the applicator and the immersion tube for transporting the gel-like substances to the reservoir.

The invention relates to a device for applying a gel-like substance, for instance a contact or ultrasonic gel, the gel-like substance being provided in a gel container.

In certain instances of medical diagnosis or therapy it is necessary to couple radiation or mechanical waves into the patient's body with a minimum of loss. Furthermore, when electrical signals are to be picked up, as in the case of recording an ECG, the contact resistance between the electrodes of the apparatus and the body surface must be kept as small as possible. In all these cases it is current practice to apply a gel layer between the patient's body surface to be treated and the contact surface of the medical apparatus. The gel layer may be applied either on the contact surface of the medical apparatus or on the body surface to be treated, it being essential that no air gap be present between the contact surface and the body surface; the gel layer itself is to be kept free of air bubbles.

According to the state of the art the contact or ultrasonic gel is kept in easily handled plastic bottles with a squirt opening or an outlet nozzle, the necessary amount of gel being applied to the body surface or the contact surface of the medical apparatus or a sensor by squeezing the plastic bottle. Since a considerable amount of gel is used in many medical applications the assisting personnel must see to it that a sufficient number of gel bottles are available or that such bottles are promptly refilled from larger storage tanks.

In addition, devices have become known which enable even unskilled clinical personnel to perform airgap-free coupling of a patient to the membrane of a treatment apparatus. From DE 35 44 710 A1, for instance, there is known a method and a device in which a gel delivering part is first placed on the membrane of the treatment apparatus, whereupon the membrane together with the part is placed on the skin of the patient. The part is then extracted from the gap between skin and membrane, with gel issuing from one or several openings of the part and completely filling the gap with gel. Such a device is too complicated yet for many applications and cannot be used for example to apply a gel layer directly on the skin surface or on the contact surface of an ultrasonic head.

Furthermore it is known from U.S. Pat. No. 5,232,498 A to apply gel to the contact surface of an ultrasonic probe by means of a roller or a continuous belt, which dips into a container filled with gel.

It is an object of the present invention to propose a device for applying a gel-like substance, for instance a contact or ultrasonic gel, which permits applying the needed amount of gel in a simple manner on the contact surface of a medical apparatus or the body surface to be treated, while avoiding personnel-intensive preparatory steps.

The invention achieves this object by proposing that the gel container be provided with an immersion tube which, departing from a docking area, extends into the gel-like substance, and that an applicator for the gel-like substance can be mounted in the docking area of the gel container, which applicator has a reservoir connected to an outlet nozzle as well as a pumping means for the gel-like substance, and that a tightly sealed connection can be established between the outlet nozzle of the applicator and the immersion tube for transporting the gel-like substance to the reservoir.

By means of the applicator the needed amount of gel can be applied without problems to the surfaces to be gel-coated. The applicator may for instance be bottle- or syringe-shaped and may be provided with a switch or a mechanism permitting exact dosage of the needed amount of gel. Once filled with the gel-like substance the applicator may be taken off the docking area on the gel container and used for applying the gel-like substance on the patient or on a medical apparatus.

According to the invention the pumping means in the applicator may be designed as a plunger pump, in which an electro-mechanically or pneumatically driven plunger is guided in a cylindrical reservoir. When the applicator sits on the gel container the plunger acts as a suction means, which when activated sucks the gel-like substance into the applicator via the immersion tube. When the gel is to be applied the plunger of the pumping means is moved in the opposite direction, causing the gel-like substance to exit from the outlet nozzle of the applicator.

The invention may be embodied in a number of variants. The plunger of the pumping means may for instance be furnished with a drive spindle connected to a motor/gear unit in the applicator by means of toothed wheels, toothed belts or the like. It will also be possible, however, to couple the drive spindle of the pumping means to a motor/gear unit positioned on or in the gel container when the applicator is placed on the container, thus avoiding a motor/gear unit in the applicator and making it light-weight and compact.

If the motor/gear unit is positioned on or in the gel container, the drive spindle or the plunger of the pumping means is provided with a spring element, which is pre-stressed by the suction movement of the plunger, and whose stored energy can be used to drive the plunger when the gel-like substance is to be applied.

According to the invention the gel container or the immersion tube may be provided with a heating device, which will heat the gel-like substance in the container to temperatures in the range of skin or body temperature, i.e. in the range of 20° C. to 40° C.

Furthermore a heating device acting on the reservoir for the gel-like substance and/or the outlet nozzle may be provided in the applicator, which is supplied with electric energy from rechargeable batteries in the applicator or via electrical contacts effective when the applicator is docked.

The invention will be further described below, with reference to the enclosed drawings.

There is shown in schematical presentation in

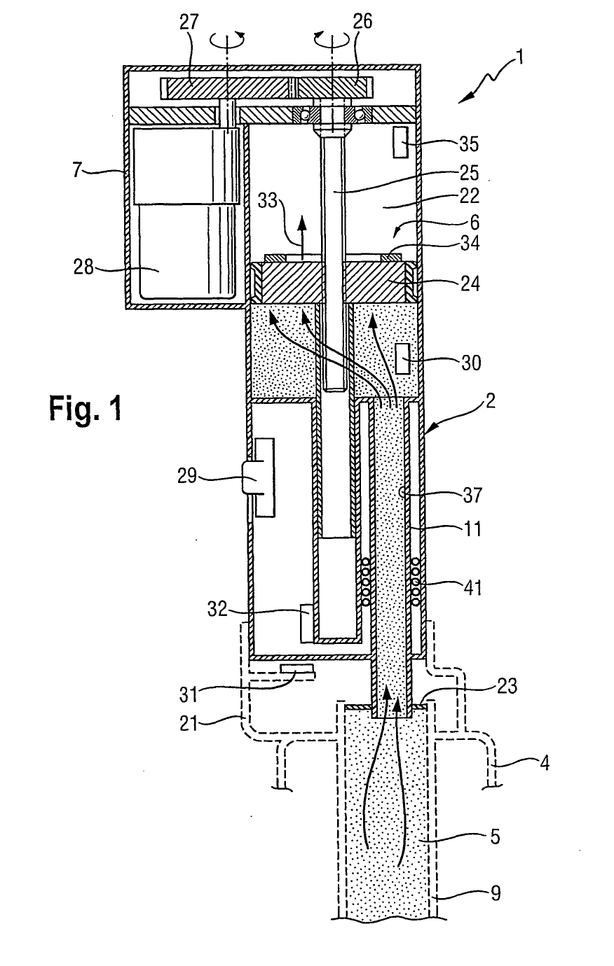

FIG. 1 a device for applying a gel-like substance according to the invention, and in

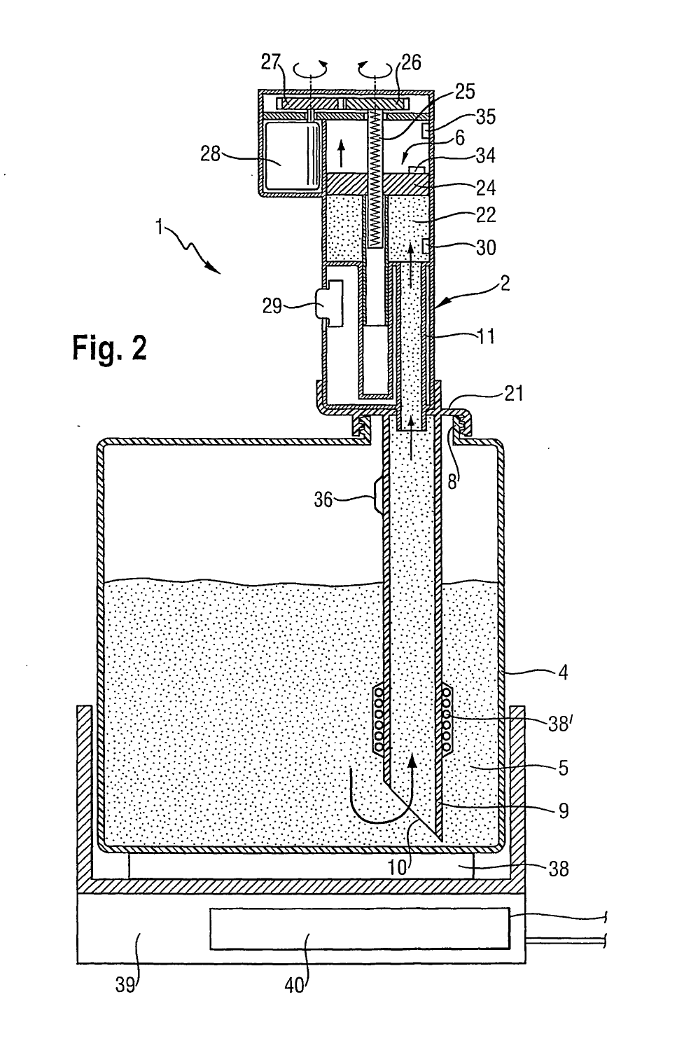

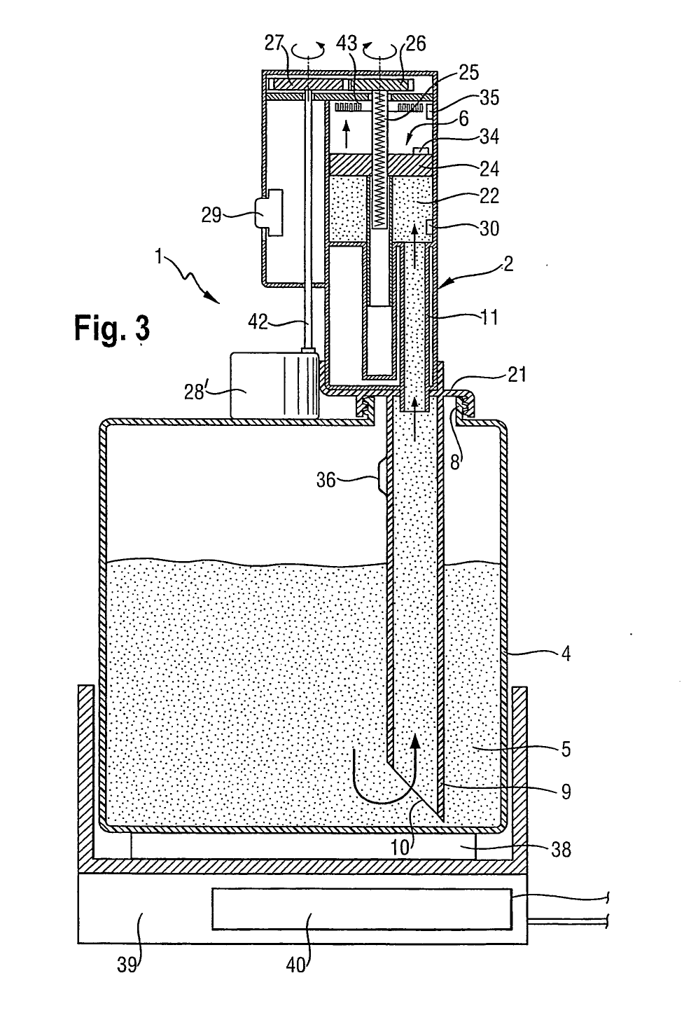

FIGS. 2 and 3 variants of the embodiment of FIG. 1.

The device 1 for applying a gel-like substance 5, for instance a contact or ultrasonic gel, as shown in FIGS. 1 and 2, has an applicator 2, which can be placed in a docking area 21 of a gel container 4 holding the gel-like substance 5.

The docking area 21 and the immersion tube 9 with its suction opening 10 (see FIG. 2) preferably form a unit, which may be directly placed or screwed onto the opening 8 of a conventional gel container 4. The applicator 2 has a reservoir 22 connected to an outlet nozzle 11, with a pumping means 6 configured as a plunger pump being provided for feeding the gel-like substance 5 into the reservoir 22. In the docked position, when the applicator 2 is placed in the docking area 21, the outlet nozzle 11 of the applicator 2 is sealed against the immersion tube 9 by means of a sealing ring 23.

In the variant shown the pumping means 6 in the applicator 2 is configured as a plunger pump, in which an electro-mechnically driven plunger 24 is guided in the cylindrical reservoir 22. Other types of pumping means, e.g. roller pumps or peristaltic pumps, could also be employed.

The plunger 24 of the pumping means 6 has a drive spindle 25, which is connected via gears 26, 27 to a motor/gear unit 28 located in the applicator 2. In the docked position the gel-like substance 5 is sucked into reservoir 22 by means of the pumping means 6 until the reservoir is completely filled. Subsequently the applicator 2 may be removed from the docking area 21 and by activating a switch 29 the gel may be applied at the desired site via the outlet nozzle 11.

The device according to the invention operates as follows:

When the applicator 2 is empty the plunger 24 rests against the bottom end of the cylindrical reservoir 22, its position being defined by the proximity end contact 30. In this state the applicator 2 is placed in the docking area 21, with the outlet nozzle 11 entering the immersion tube 9 and being sealed against ambient air by the sealing ring 23.

According to the invention magnetic switching elements 31, 32 (e.g. reed contacts) are provided in the docking area 21 and at the end of the applicator 2, which automatically control the intake of the gel-like substance 5 in the docked position of the applicator 2. By the magnetic proximity switch 31 the motor of the motor/gear unit 28 is activated, such that via gears 26, 27 the drive spindle 25 moves the plunger 24 in the direction of arrow 33.

Due to the negative pressure generated in the outlet nozzle 11 the gel-like substance 5 is sucked from the immersion tube 9, or rather from the gel container 4, into the reservoir 22 of the applicator 2.

According to the invention the plunger 24 of the pumping means 6 and the cylindrical reservoir 22 for the gel-like substance 5 are provided with magnetic switching elements 34, 35 which automatically terminate the loading process when they approach each other. The sucking-in process thus continues until either the applicator is removed from the docking area (switch 31 opens) or until the switching element 35 cuts off motor current upon approach of the switching element 34 affixed to the plunger 24.

After removal of the applicator 2 from the docking area 21 by activating the switch 29 the gel contained in the reservoir 22 may through the outlet nozzle 11 be applied at the desired site by means of the plunger 24. Applying the gel-like substance continues until the switching element 34 on the plunger 24 reaches the end contact 30 and stops the motor. The applicator may now be filled again as described above.

When a new container 4 is used the still empty immersion tube 9 must first be inserted into the container 4. Usually the new immersion tube 9 will only partly be filled by the gel-like substance 5. Since in most cases the volume of the reservoir 22 of the applicator 2 is smaller than the volume of the immersion tube 9, repeated sucking-in and expelling (of air) will be required before the reservoir 22 is filled with the gel-like substance 5. An automated procedure may be provided for this case, in which air from the immersion tube 9 is cyclically sucked in and expelled via a valve 36 on the immersion tube 9 (see FIG. 2) until the applicator 2 sucks in gel and the reservoir 22 is being filled with the gel-like substance. To determine whether gel or air is being sucked in an optical sensor 37 may be provided in the outlet nozzle 11.

To clean the applicator 2 a certain number of sucking and expelling cycles may be activated, with the outlet nozzle 11 of the applicator 2 dipped into a cleaning fluid (for instance warm water).

As shown in FIGS. 2 and 3 the gel container 4 may be provided with a heating device 38, which heats the gel-like substance 5 in the container 4 to temperatures in the range of 20° C. to 40° C. The heating device 38 may be part of a frame 39 holding the gel container 4, which frame 39 also contains a power supply unit 40 for the motor/gear unit 28. A heating device 38′ may also be directly attached to the immersion tube 9 (see FIG. 2) to heat only the amount of gel to be sucked in by the applicator.

A heating device 41 may also be provided in the applicator 2, acting on the reservoir 22 or the outlet nozzle 11.

According to the invention electrical power may be supplied to the docked-on applicator 2 by automatically established electrical contacts between applicator 2 and docking area 21. Furthermore, power can be supplied to the applicator 2 by means of a power supply cable, which will guarantee direct energy supply, for instance for the heating device 41 in the applicator 2, even when the applicator has been removed from the docking area.

The applicator 2 may also contain rechargeable batteries, which are charged when the applicator 2 is in the docking area and supply electric energy for driving the pumping means 6 and possibly for the heating unit 41 when the applicator is removed from the docking area.

The switch 29 for dispensing the gel-like substance 5 may be an on/off switch, a stepping switch or a continuous controller determining the amount of gel-like substance 5 to be applied per unit of time.

In the variants shown the plunger 24 is driven by the drive spindle 25, which is fixed in axial direction and moves the plunger 24 in axial direction by means of an interior thread in the plunger 24. Other driving designs could be considered, such as via an interior thread in the gear 26 or a pneumatic plunger drive by means of a pressured air cartridge in the applicator.

The variant according to FIG. 3 differs from that of FIG. 2 insofar as the motor/gear unit 28′ is placed on the gel container 4, with a drive shaft 42 connecting to the motor/gear unit 28′ when the applicator 2 is placed in the docking area 21. In this variant a spring element 43 is stressed during the loading process by the drive spindle 25 or the plunger 24 of the pumping means 6, which moves the plunger 24 in output direction when the gel-like substance is applied. The spring element 43 may for instance be configured as a spiral spring.

1.-11. (canceled)

12. A device for applying a gel-like substance, comprising:
a gel container for containing said gel-like substance comprising an immersion tube departing from a docking area and extending into the gel-like substance;
an applicator for the gel-like substance being mountable in the docking area of the gel container, said applicator having a reservoir connected to an outlet nozzle as well as a pumping means for transporting the gel-like substance;
wherein a tightly sealed connection is established between the outlet nozzle of the applicator and the immersion tube for transporting the gel-like substance to the reservoir.

13. A device according to claim 12, wherein the pumping means in the applicator is configured as a plunger pump, in which an electromechanically or pneumatically driven plunger is guided in a cylindrical reservoir.

14. A device according to claim 13, wherein the plunger of the pumping means has a drive spindle, which is connected to a motor/gear unit located in the applicator.

15. A device according to claim 13, wherein the plunger of the pumping means has a drive spindle, which connects to a motor/gear unit located in or on the gel container when the applicator is placed in said docking area of the gel container.

16. A device according to claim 15, wherein the drive spindle or the plunger of the pumping means cooperates with a spring element, which is stressable when the applicator is placed in said docking area of said gel container.

17. A device according to claim 12, wherein the gel container or the immersion tube is provided with a heating means for heating the gel-like substance in the gel container or in the immersion tube to a temperature in the range of 20° C. to 40° C.

18. A device according to claim 12, wherein the applicator is supplied with electrical power via electrical contacts on the applicator and on the gel container, which are automatically effective when the applicator is placed in said docking area of said gel container.

19. A device according to claim 12, wherein the applicator has a heating means acting on the reservoir for the gel-like substance and/or the outlet nozzle.

20. A device according to claim 12, wherein the applicator is provided with rechargeable batteries, which are charged via electrical contacts automatically effective when the applicator is placed in said docking area of said gel container.

21. A device according to claim 12, wherein magnetic switching elements are provided in the docking area and in the applicator, which automatically control the intake of the gel-like substance when the applicator is placed in said docking area of said gel container.

22. A device according to claim 21, wherein magnetic switching elements are provided on the plunger of the pumping means and in the cylindrical reservoir for the gel-like substance, which automatically terminate the loading process and the expelling process.

23. A device according to claim 12, wherein the gel-like substance is a contact or ultrasonic gel.
